(19) United States
(12) Patent Application Publication
(10) Pub. No.: US 2024/0299612 A1
(43) Pub. Date: Sep. 12, 2024

(54) Method and Device for Disinfecting the Air Breathed in by a Person

(71) Applicant: Jean-Pierre Brune, La Valette (FR)

(72) Inventors: Jean-Pierre Brune, La Valette (FR); Magali Salesco Brune, Sollies-Pont (FR); Caroline Brune, Sollies-Pont (FR)

(21) Appl. No.: 18/546,635

(22) PCT Filed: Feb. 2, 2022
(86) PCT No.: PCT/FR2022/050201

§ 371 Date: Aug. 16, 2023

(30) Foreign Application Priority Data
Feb. 15, 2021 (FR) 2101409

(51) Int. Cl. A61L 9/20; A62B 18/00; A62B 18/02

(52) U.S. Cl. CPC A61L 9/20; A62B 18/006; A62B 18/025; A61L 2209/111; A61L 2209/12

(57) ABSTRACT

A method for disinfecting the air breathed in by a user includes the steps of: —subjecting the inside of a first and a second chamber in communication with an outside air space to a first source of ultraviolet radiation having a wavelength suitable for destroying germs, —detecting air inhalation and exhalation breathing phases carried out by the user, each breathing phase being associated with a forward or backward direction in which air is circulated in a pipe during the breathing phase, —during a first forward direction breathing phase, circulating the air from the first chamber between an internal space of a breathing mask worn by the user and the outside air space, and—during a second forward direction breathing phase, circulating the air from the second chamber between the internal space of the breathing mask and the outside air space.

### BACKGROUND OF THE INVENTION

### 1. The Field of the Invention

The present invention relates to an air disinfecting device associated with an oral-nasal respiratory mask for protection against infections, and a method for disinfecting the air breathed in by a person.

### 2. The Relevant Technology

Ultraviolet rays (UV) and in particular those located in the UV-C wavelength band (240 to 280 nm) act against viruses and bacteria by killing them or making them incapable of infecting a person. This is why UV lamps are found in numerous air and water purification devices, or in chambers for disinfecting objects, such as glasses or telephones.

It has also been proposed to integrate one or more UV lamps into an oral-nasal respiratory mask for protection against infections, to eradicate viruses and bacteria. It turns out that the light intensity of the emitted UV radiation and the UV exposure time are insufficient to have a real effect on the air breathed in through a respiratory mask. In reality, the ultraviolet rays emitted only have an effect on the germs present on the surface of the mask.

The doses of ultraviolet rays necessary to eradicate the viruses and bacteria in an air volume depend on several factors, namely the wavelength and the light intensity of the received UV radiation, as well as the exposure time, and more precisely, the dose received which is equal to the product of the light intensity of the UV radiation multiplied by the exposure time. In addition, it is generally accepted that the survival of the germs when exposed to ultraviolet rays follows an exponential decay model, of the shape S=e^{-kD}, where S represents the fraction of germs still present after exposure to ultraviolet radiation, D represents the applied dose, and k a coefficient that is specific to each germ.

Moreover, the breathing flow rates vary in large proportions depending on whether the person is at rest or engaging in physical activity, and from one person to another.

It is therefore desirable to propose a device that is effective at disinfecting a very variable volume of air breathed in or out by a person. It is also desirable for this device to be easily carried by a person, without impeding their daily movements.

### SUMMARY OF THE INVENTION

Embodiments relate to a method for disinfecting the air breathed in by a user, the method comprising steps consisting in: —subjecting the inside of a first chamber in communication with an outside air space to a first source of ultraviolet radiation having a wavelength suitable for destroying germs, subjecting the inside of a second chamber in communication with the outside air space, to a second source of ultraviolet radiation, detecting the moments when the inhalation and exhalation breathing phases carried out by the user start, each breathing phase being associated with a forward or backward direction in which air is circulated in a pipe during the breathing phase, during a first forward direction breathing phase following a first backward direction breathing phase, circulating the air from the first chamber between an internal space of a respiratory mask sealingly covering the mouth and the nose of the user and the outside air space, the first forward direction breathing phase being followed by a second backward direction breathing phase, and during a second forward direction breathing phase following the second backward direction breathing phase, circulating the air from the second chamber between the internal space of the breathing mask and the outside air space.

According to one embodiment, the method comprises a step of detecting that the air of the first chamber is fully changed before the end of the first forward direction breathing phase, followed by a step of circulating the air of the second chamber alternately with the circulating of the air from the first chamber, between the internal space of the respiratory mask and the outside air space.

According to one embodiment, the method comprises several alternating steps to circulate the air from the first chamber and to circulate the air from the second chamber, between the internal space of the respiratory mask and the outside air space, during the first forward direction breathing phase, each alternation being triggered by the detection that the air is fully changed in the first or second chamber.

According to one embodiment, the method comprises steps of turning off the first and second ultraviolet radiation sources during each backward direction breathing phase.

According to one embodiment, the first and second forward direction breathing phases are user inhalation phases, and the first and second backward direction breathing phase are user exhalation phases, or the first and second forward direction breathing phases are user exhalation phases, and the first and second backward direction breathing phases are user inhalation phases.

According to one embodiment, the method comprises steps of activating a fan to circulate the air during each forward direction breathing phase.

Embodiments may also relate to a device for disinfecting the air breathed in by a user, the device comprising: a first chamber and a second chamber, in communication with an external volume of air and coupled by a controlled valve device, to a first pipe connected to the internal space of a respiratory mask, an ultraviolet source, installed inside each of the first and second chambers, and a measuring device for determining a direction in which the air circulates in the first pipe, the disinfecting device being configured to implement the method defined above, the detection of the moments when the inhalation and exhalation breathing phases start being carried out using the measuring device, and the circulating of the air in the first chamber and in the second chamber being carried out using the valve device.

According to one embodiment, each of the first and second chambers comprises a ventilation device for circulating the air through the chamber.

According to one embodiment, the ventilation device comprises a pump or a fan and/or implements the Coanda effect.

According to one embodiment, the source of UV radiation in each of the first and second chambers comprises a UV quartz tube or UV LED lamp.

According to one embodiment, the device is installed in a portable housing, including an autonomous electrical power source.

According to one embodiment, each of the first and second chamber is associated with a compressor to increase the pressure in the chamber.

According to one embodiment, the UV radiation source is installed in each of the first and second chambers and configured to subject an exposure volume in the chamber to a light intensity greater than or equal to 10 mW/cm².

### BRIEF DESCRIPTION OF THE DRAWINGS

Exemplary embodiments of the invention will be described in what follows, by way of non-limiting example in relation to the accompanying figures, among which:

FIG. 1 schematically shows a disinfecting device, according to one embodiment,

FIG. 2 schematically shows an element of the disinfecting device, according to one embodiment,

FIGS. 3 to 5 are schematic timing diagrams of variation of the flow rate of breathable air, illustrating the operation of the disinfecting device, according to various embodiments.

### DETAILED DESCRIPTION OF THE PREFERRED EMBODIMENTS

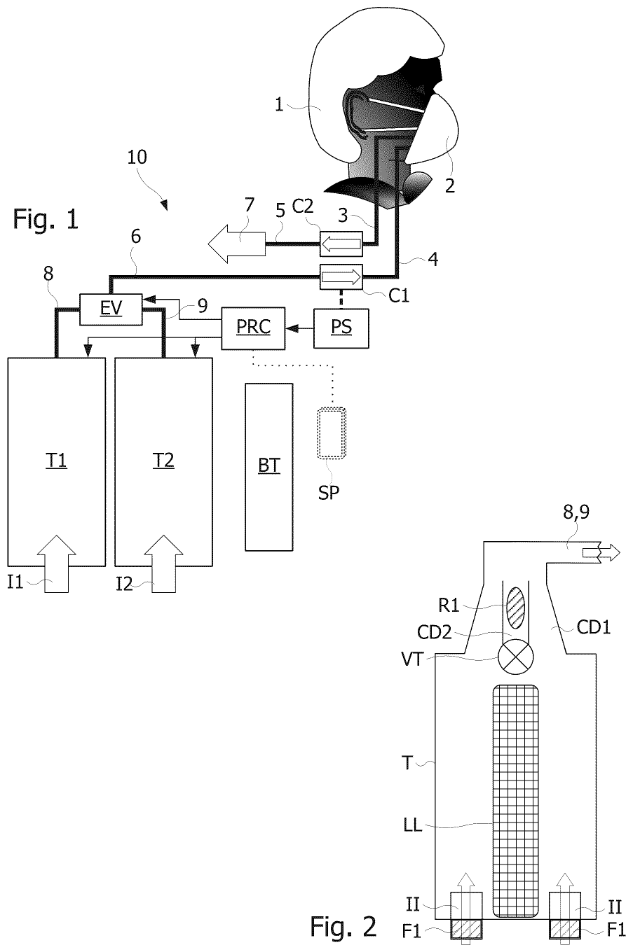

FIG. 1 shows a device 10 for disinfecting air breathed in, according to one embodiment. The device 10 comprises two chambers T1, T2, an electrically controlled valve device EV, a pressure sensor PS, two check valves C1, C2, and a processor PRC receiving pressure measurement signals from the pressure sensor PS and controlling the valve device EV and the chambers T1, T2. The device 10 also comprises a sealed oral-nasal respiratory mask 2, arranged to be attached to the head of the user 1 so as to sealingly cover the mouth and nose of the user. The internal space of the mask 2 is connected to the valves C1, C2 by two pipes 3, 4. The valve C2 is connected to an inlet 7 of the device by a pipe 5, the inlet 7 being open to the ambient air. The valve C2 can be integrated directly into the mask 2. The valve C1 is connected to the valve device EV via a pipe 6, the device EV being connected to the chambers T1, T2 by respective pipes 8, 9. The pressure sensor PS measures the pressure in the pipe 4 for example in the valve C1. The valve device EV is arranged so as to connect the pipe 6 either to the pipe 8 leading to the chamber T1, or to the pipe 9 leading to the chamber T2. Each chamber T1, T2 comprises an inlet 11, 12 open to the ambient air, and a source of UV radiation (FIG. 2). Each chamber T1, T2 may also comprise a fan to facilitate the circulation of air in the device and avoid forcing the user 1 to breathe harder. The device 10 also comprises one or more batteries BT for powering the processor PRC, the sensor PS, the device EV and the chambers T1, T2 (in particular the source of radiation).

The set of chambers T1, T2, the battery BT, the valve device EV and the processor PRC, can be arranged in a housing (box or bag) associated with fastening means for suspending the housing to the neck or shoulders of the user 1, or for holding it around the waist of the user. The battery BT can be arranged in a separate housing, for example fixed to the user's waist.

When the user 1 exhales air, the valve C2 opens, while the valve C1 remains closed. The exhaled air is therefore expelled through the opening 7. When the user inhales air, the valve C1 opens, while the valve C2 remains closed. The processor PRC commands the device EV to place the pipe 6 in communication with the chamber T1 and the chamber T2, in alternating fashion. Thus, the inhaled air comes either from the chamber T1, or from the chamber T2, which fills gradually, through its inlet 11, 12.

The direction of circulation of the air in the pipes 6, 8 and 9, and in the chambers T1, T2 can be reversed, for example to disinfect the air exhaled by the user 1, by reversing the connections of the pipes 5, 6 to the valves C1, C2.

The pressure sensor PS may be a piezoresistive sensor integrated into a semiconductor component comprising a gas inlet and outlet, and supplying an analog voltage proportional to the flow rate of gas between the inlet and the outlet. The sensor PS can be connected in shunt with the valve C1. The pressure sensor PS may also comprise a impeller or a propeller arranged in the pipe 4 or 6 and associated with a device for measuring the speed of the impeller or propeller. The measurement of this speed makes it possible to determine the speed of the airflow in the pipe and therefore the flow rate of the air.

The valve device EV may be of the three-way, two-position solenoid valve type. Thus, the solenoid valve may comprise an electromagnet which, at rest, maintains the open connection toward the chamber T1 (connection between the pipes 6 and 8 is open and connection between the closed pipes 6 and 9 is closed), and under tension, keeps the connection open to the chamber T2 (connection between the pipes 6 and 9 is open and connection between the pipes 6 and 8 is closed). The valve device EV may comprise two solenoid valves with two channels arranged respectively between the pipes 6 and 8 and between the pipes 6 and 9. These two solenoid valves can be mounted so as to open the connection between the two pipes 6, 8 or 6, 9 when they are supplied, and close the connection in the opposite case.

The check valves C1, C2 may comprise a mobile valve or be of the Tesla valve type allowing a fluid to flow in only one direction without a moving part.

The processor PRC may be of the microprocessor or microcontroller type.

The mask 2 comprises an air-tight shell that sealingly covers the user's mouth and chin. The shell may be rigid, transparent and thin, for example less than or equal to 2 mm. The shell may be manufactured from a rhabdoid sheet. The sealing between the shell and the face of the user 1 can be obtained via a flexible elastomer joint. To ensure good sealing, the shape of the shell may be fitted to the shape of the user's face. The shell can be held onto the user's face by elastic strips passing behind the ears or behind the head, or by eyeglass temples.

The pipes 3, 4 can open into the shell or be coupled to a single flexible pipe opening into the shell, and for example attached to one of the eyeglass temples. The pipes 3, 4 or the single pipe can open into the mask by an opening made at the cheeks by a double skin leaving a passage several millimeters wide.

The fitting of the shape of the shell to the shape of the user's face can be carried out using a 3D model of the face. The 3D model can be used to select a mask from a set of prefabricated masks, a final fitting of the prefabricated mask being able to be carried out for example by thermoforming. Alternatively, the mask can be manufactured from a mold produced for example by means of plaster strips placed on the face.

The mask may be equipped with a microphone connected to an amplifier coupled to a loudspeaker, to compensate for the fact that the mask may muffle the voice. When the user speaks, their respiratory flow rate may be disturbed. To compensate, the processor PRC may be connected to the microphone.

FIG. 2 shows a chamber T, according to one embodiment, the chambers T1, T2 potentially being identical to the chamber T. The chamber T has an elongated shape and comprises, at one end, an opening I1 open to the ambient air, which can be equipped with a filter F1, at an opposite end, a transition zone CD1 between the chamber T and the pipe 8 or 9, and between these two ends, an exposure zone of the air present in the ultraviolet chamber, the exposure zone comprising a source LL of UV radiation. The shape of the chamber T is adapted to the shape of the source LL so as to ensure substantially uniform illumination of the space of the exposure zone. The inner walls of the chamber can be covered with a reflective coating. The transition zone CD2 may comprise a fan VT facilitating inhalation by the user 1, the speed of the fan being controlled by the processor PRC as a function of the pressure measured by the pressure sensor PS.

According to one embodiment, the fan VT is arranged in or at the end of a pipe CD2 attached coaxially in the transition zone CD1, the inside of the pipe CD1 being partially blocked by an ellipsoid-shaped part R1 attached coaxially in the pipe CD1. The air that leaves the exposure zone of the chamber T is sucked by the fan VT into the pipe CD2 and the air which passes through the transition zone CD1 around the pipe CD2 produces a multiplier effect by the Coanda effect, which makes it possible to obtain a large air flow at the outlet of the chamber in the pipe 8, 9, even with a low-power fan.

In the example of FIG. 2, the source LL has a cylindrical shape. Thus, the source LL may, for example, be of low-pressure quartz tube type producing radiation centered on 254 nm (exactly 253.7 nm), these wavelengths having a germicidal action. In addition, the quartz crystal has the advantage of filtering the wavelength of 185 nm which reacts with the oxygen in the air to produce ozone, which is toxic. The exposure zone of the chamber T may also have a cylindrical shape, the source LL being attached coaxially to the exposure zone in the chamber T. The opening II, as well as the filter F1 may then have an annular shape around the source LL, as shown in FIG. 2.

The chamber T may have any other shape, in particular a shape suitable for its transport, for example a parallelepiped shape. In addition, the chamber T may have several openings. Moreover, baffles can be provided around opening I1 in order to prevent UV radiation from exiting chamber T, as UV-C is hazardous to the eyes and skin.

The source LL may also be based on light-emitting diodes (LEDs), the number and the arrangement of the LEDs in the exposure zone and the shape of the latter being adapted accordingly to obtain substantially uniform illumination of the space of the exposure zone. UV LEDs can produce radiation centered on 265 nm. UV LEDs tending to clear much heat, they may be coupled to a heat pipe or a Peltier effect module, or be associated with a fan.

The VT fan can be replaced by or associated with a single pump for both chambers T1, T2, for example one placed in the pipe 6. The inlet I1 of each of the two chambers T1, T2 can be equipped with a check valve.

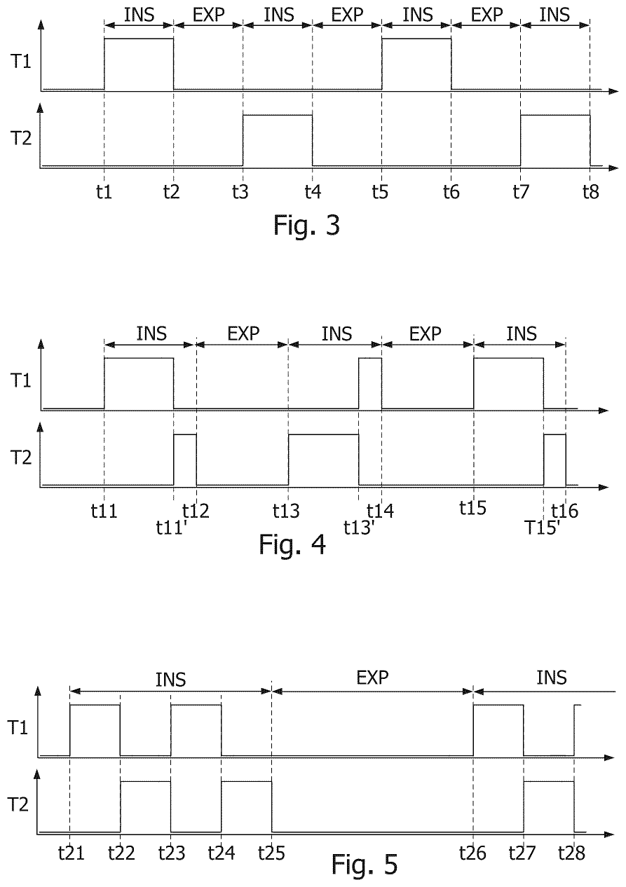

FIGS. 3 to 5 are timing diagrams of the variation of the air flow rate breathed in the air disinfecting device 10. FIG. 3 shows more particularly the case where the space of air present in each of the chambers is sufficient to supply inhalation. FIG. 3 shows inhalation phases INS bounded by the times t1-t2, t3-t4, t5-t6 and t7-t8 alternating with the user's expiration phases EXP, delimited by the times t2-t3, t4-t5, t6-t7.

Before the time t1, the valve EV of check valve C1 is closed. The two chambers T1, T2 are therefore not in communication with the mask 2, the air in the chambers being subjected to UV radiation. At time t1, the user starts to inhale air, which triggers the opening of the check valve C1. The processor PRC then detects a negative pressure (pressure measurement less than a first threshold value) and therefore commands the valve device EV to place the pipes 6 and 8 in communication (chamber T1), and triggers the fan VT to draw the air into the chamber T1 at a speed adjusted as a function of the pressure measurements provided by the sensor PS.

Between the times t2 and t3 (as well as at each expiration phase EXP), the user exhales air, which closes the check valve C1 and opens the check valve C2. The pressure in the pipe 4 passes above a second threshold value. The processor PRC then commands the fan VT in the chamber T1 to stop.

At time t3, the processor PRC again detects that the user inhales air by means of the sensor PS, and commands the valve device EV to place the pipes 6 and 9 in communication (chamber T2). The processor PRC also turns on the fan VT as a function of the pressure measured by the sensor PS. Thus, the inhalation phases INS are carried out alternately through the chambers T1, T2. As a result, during the period between the times t2 and t5 extending over two expiration phases EXP and an inhalation phase INS, the air in the chamber T1 is not fully changed and therefore remains exposed to UV. Likewise, during the period between the times t4 and t7 extending over two expiration phases EXP and an inhalation phase INS, the air in the chamber T2 is not fully changed and remains exposed to UV.

According to one embodiment, the source LL of UV radiation is a low-pressure quartz tube having a length of 12.5 cm and a diameter of 3 cm, and emitting a light power of 3 W. The light intensity at the surface of the tube can be estimated at 8.4 mW/cm². If the chamber T1, T2 has a volume of 0.5 L excluding the volume of the tube and with the tube being positioned at the center of the chamber, then the light intensity of the UV radiation applied to the volume of air in the chamber T1, T2 is about 10 mW/cm².

The efficacy of ultraviolet against a germ is generally measured by the dose D₉₀ necessary to eradicate 90% of the germ. This value depends on the nature of the germ and the medium in which the germ is present. Different studies show that viruses and bacteria are very UV-sensitive, especially viruses in dry air and a little less so in humid air. According to various authors, coronaviruses require doses ranging from 7 to 2410 J/m² to achieve a 90% reduction. Studies on the SRAS-COV-2 virus (Bianco et al., “UV-C irradiation is highly effective in inactivating SARS-COV-2 replication”, 2020, doi https://doi.org/10.1101/2020.06.05.20123463) establish that the average value of the Dgo dose is 2.7 mJ/cm², and suggest that a dose equal to 4.7 mJ/cm² would be effective against all coronaviruses.

A person at rest inhales and exhales on average 0.5 L and carries out 12 to 16 respiratory cycles per minute. This results in a breathing flow rate of 8 L/min and a duration of a minimum respiratory cycle of 3.8 s. An inhalation or exhalation (between the times ti and ti+1, where i=1, 2, 3, . . . ) therefore lasts 1.9 s. The chambers T1 and T2 can therefore have a volume at least equal to 0.5 L. The air in each chamber T1, T2 therefore remains exposed to UV for three times 1.9 s, or 5.7 s, omitting any time intervals between the respiratory and exhalation breathing phases.

A light intensity of 10 mW/cm² makes it possible to achieve a dose of 57 mJ/cm² during an exposure phase (between the times t2 and t5 or between the times t4 and t7) of 5.7 s, that is a value much greater than the dose of 4.7 mJ/cm² effective for removing 90% of coronaviruses. The law of variation of the number of germs as a function of time is logarithmic, so doubling of the exposure time makes it possible to destroy ten times more germs.

FIG. 4 shows the operation of the air disinfecting device 10 when the physical activity of the user 1 is more sustained, that is, when the volume of air inhaled at each inhalation INS exceeds the volume of air in each of the chambers T1, T2. In the example of FIG. 4, the volume of each chamber T1, T2 is 0.5 L, the respiratory rate of the user is 12 L/min, reached at 17 cycles of breathing per minute. The volume of air through inhalation is therefore 0.7 l and the inhalation INS or expiration period EXP is 1.76 s. Each inhalation INS and expiration phase EXP extends between times ti and ti+1, (i=11, 12, 13, 14, 15).

Before the time t11, the air present in the chambers T1, T2 is disinfected, and the valve C1 is in its closed position. At time t11, the valve device EV is controlled to place the chamber T1 in communication with the pipe 6. Between the times t11 and t11′, the air present in the chamber T1 is totally drawn in. At time t11, the processor PRC determines that all the disinfected air in the chamber T1 was drawn as a function of the pressure measurement provided by the sensor PS (which makes it possible to calculate the flow rate and therefore the volume drawn in), and commands the valve device EV to place the chamber T2 in communication with the pipe 6. Between the times t11′ and t12, the inhalation phase continues, a part of the disinfected air present in the chamber T2 being inhaled by the user. Some of the air present in the chamber T2 was therefore changed between the times t11′ and t12. The user is in the expiration phase EXP between the times t12 and t13. In the following inhalation phase INS, between the times t13 and t14, the processor PRC commands the valve device EV to provide the user 1 with all of the disinfected air present in the chamber T2, then some of the disinfected air present in the chamber T1, between the times t13′ and t14. Then, the device 10 is in the expiration phase EXP between the times t14 and t15. Between the time t15 and t16, the device returns again to an inhalation phase INS which takes place in the same way as between the times t11 and t12.

The exposure time to ultraviolet air in each of the chambers T1, T2 is greater than three inhalation or exhalation phases (between the times t11′ and t15 for the chamber T1), that is 5.3 s. However, during these three phases, some of the air (0.2 L) was changed in the chamber T1, between the times t13′ and t14, and was exposed to the UV for a single expiration phase EXP, between the times t14 and t15. That part of the changed air therefore received a dose of 17.6 mJ/cm² (10×1.76).

When the remaining volume to be drawn in is low at time t11′, given the preceding breathing phase, it may be provided to leave the chamber T1 in communication with the pipe 6, knowing that the air that enters through the orifice I1 at the base of the chamber T1 passes through the entire length thereof and is therefore subjected to UV.

FIG. 5 shows the operation of the air disinfecting device 10 when the physical activity of the user 1 is even more sustained. In the example of FIG. 5, the user 1 performs 30 respiratory cycles per minute and in turn 60 L/min (that is seven times the volume at rest). Each inhalation and exhalation phase, between the times t21 and t25, and the times t25 and t26, therefore lasts 1 s, and the volume of inhalation air or exhaled at each inhalation or exhalation is 2 L.

Between the times t21 and t22, all of the air of the chamber T1 is drawn in. At time t22, the processor PRC determines that all the disinfected air from the chamber T1 was drawn as a function of the pressure measurement provided by the sensor PS, and commands the valve device EV to place the chamber T2 in communication with the pipe 6. At time t23, the processor PRC determines that all the disinfected air of the chamber T2 has been drawn in, and commands the valve device EV to place the chamber T1 in communication with the pipe 6. This process is repeated once between the times t23 and t25 to reach the end of the inhalation phase INS. Thus, between the times T21 and t25, the device 10 carries out four inhalation phases carried out alternately in each chamber T1, T2, to provide the 2 L inhaled by the user, during the inhalation phase INS of duration 1 s. Each inhalation phase between the times ti and ti+1 (i=21, 22, . . . 24), carried out during the inhalation phase INS therefore lasts 0.25 s. The expiration phase EXP extends over a second between the times t25 and t26, the exposure phase of the chamber T1 taking place between the times t24 and t26, and the exposure phase of the chamber T2 taking place between the times t25 and t27. The process run during the times t21 to t25 is run again beginning at the time t26.

In the preceding example, wherein the light source LL produces a light intensity of 10 mW/cm², an exposure time of 0.5 s makes it possible to achieve a dose of 5 mJ/cm² which is even greater than the dose of 4.7 mJ/cm² effective for eliminating 90% of coronaviruses.

In the case where the direction wherein the air circulates in the pipes 6, 8 and 9, and in the chambers T1, T2 is reversed, the inhalation INS and exhalation phases EXP in FIGS. 3, 4 and 5 are reversed.

According to one embodiment, the air introduced into the chambers T1, T2 is compressed by means of an air compressor, to increase the disinfection capacity of the device, without increasing the volume thereof, or reducing this volume while maintaining the same air disinfecting capacity. The air compressor can be coupled to the inlets 11, 12 of the chambers T1, T2 in the case where the device is used to disinfect the inhaled air. In this case, the valve device EV is configured to be able to close the pipes 8 and 9 at the same time leading to the chambers T1, T2. The air compressor can be coupled to the pipes 8, 9 connected to the chambers T1, T2 in the case where the device is used to disinfect the exhaled air, the inlets 11, 12 being equipped with check valves or controlled valves to keep the chambers T1, T2 closed when the compressor raises the pressure in the chambers.

The processor PRC can be coupled to a communication circuit, for example of the Bluetooth type, to put the processor into communication with an external SP terminal such as a mobile telephone. The processor PRC can then be configured to transmit data to the terminal SP, such as measurements relating to the breathing of the user (volumes, flow rate, frequency, etc.), theoretical percentages of germs destroyed, a state of charge of the battery BT, a number of hours of use of the device, etc.

It will be apparent to those skilled in the art that the present invention is capable of various alternative embodiments and various applications. In particular, the invention is not limited to a device wherein the UV radiation sources are permanently active. Indeed, it may be desirable to extinguish the sources of UV radiation during phases where air is not renewed in chambers T1, T2, that is during inhalation phases in the example of FIG. 2, or during the exhalation phases when the direction in which the circulates air in the chambers is reversed to disinfect the air exhaled by the user. Indeed, certain sources such as LEDs tend to heat up. It may also be desirable to prolong the duration of use of the device without having to change or recharge the battery BT. In addition, it may be sufficient to expose the air in the chambers to UV radiation only during the inhalation or exhalation phases. The light power emitted by the source LL during the duration of each of the inhalation or exhalation phases may be sufficient to destroy the germs targeted as a function of the air flow rate.

The air disinfecting device may comprise more than two chambers in parallel, in particular in order to be able to treat a greater flow of air.

1. A method for disinfecting the air breathed in by a user, the method comprising steps of:
subjecting the inside of a first chamber in communication with an outside air space, to a first source of ultraviolet radiation having a wavelength able to destroy germs,
subjecting the inside of a second chamber in communication with the outside air space, to a second source of ultraviolet radiation,
detecting times when the air inhalation and expiration breathing phases carried out by the user start, each breathing phase being associated with a forward or backward direction of circulation in which air is circulated in a pipe during the breathing phase,
during a first forward direction breathing phase following a first backward direction breathing phase, circulating the air from the first chamber between an inner volume of a breathing mask covering the mouth and the nose of the user and the outside air space, the first forward direction breathing phase being followed by a second backward direction breathing phase, and
during a second forward direction breathing phase following the second backward direction breathing phase, circulating the air from the second chamber between the internal space of the breathing mask and the outside air space.

2. The method according to claim 1, comprising a step of detecting that the air of the first chamber is fully changed before the end of the first forward direction breathing phase, followed by a step of circulating the air of the second chamber alternately with the circulating of the air from the first chamber, between the internal space of the respiratory mask and the outside air space.

3. The method according to claim 1, comprising several alternating steps to circulate the air from the first chamber and to circulate the air from the second chamber, between the internal space of the respiratory mask and the outside air space, during the first forward direction breathing phase, each alternation being triggered by the detection that the air is fully changed in the first or second chamber.

4. The method according to claim 1, comprising steps of turning off the first and second ultraviolet radiation sources during each backward direction breathing phase.

5. The method according to claim 1, wherein:
the first and second forward direction breathing phases are user inhalation phases, and the first and second backward direction breathing phase are user expiration phases, or
the first and second forward direction breathing phases are user exhalation phases, and the first and second backward direction breathing phase are user inhalation phases.

6. The method according to claim 1, comprising steps of activating a fan to circulate the air during each forward direction breathing phase.

7. A device for disinfecting air breathed in by a user, the device comprising:
a first chamber and a second chamber, in communication with an outside air volume and coupled by a controlled valve device, to a first pipe connected to the internal space of a breathing mask,
an ultraviolet radiation source installed inside each of the first and second chambers, and
a measurement device for determining a direction in which air circulates in the first pipe, the disinfecting device being configured to implement the method according to claim 1, the detection of the times when the air inhalation and exhalation phases start being carried out using the measuring device, and the circulating of the air in the first chamber and in the second chamber being carried out using the valve device.

8. The device according to claim 7, wherein each of the first and second chambers comprises a ventilation device for circulating the air through the chamber.

9. The device according to claim 8, wherein the ventilation device comprises a pump or a fan and/or implements the Coanda effect.

10. The device according to claim 7, wherein the source of UV radiation in each of the first and second chambers comprises a UV quartz tube or UV LED lamp.

11. The device according to claim 7, wherein the device is installed in a portable housing, including an autonomous electric power source.

12. The device according to claim 7, wherein each of the first and second chamber is associated with a compressor to increase the pressure in the chamber.

13. The device according to claim 7, wherein the UV radiation source is installed in each of the first and second chambers and configured to subject an exposure volume in the chamber to a light intensity greater than or equal to 10 mW/cm².
